(19) United States
(12) Patent Application Publication
(10) Pub. No.: US 2024/0000308 A1
(43) Pub. Date: Jan. 4, 2024

(54) METHODS AND APPARATUS FOR PERFORMING ENHANCED FULL-FIELD STIMULUS THRESHOLD (FST) TESTS

(71) Applicant: Diagnosys LLC, Lowell, MA (US)

(72) Inventors: Jeffrey D. Farmer, Chelmsford, MA (US); Bruce Doran, Westford, MA (US); Richard Robson, Holt (GB)

(21) Appl. No.: 18/037,818

(22) PCT Filed: Nov. 22, 2021
(86) PCT No.: PCT/US21/60379

§ 371 Date: May 19, 2023

Related U.S. Application Data
(60) Provisional application No. 63/116,336, filed on Nov. 20, 2020.

(51) Int. Cl. A61B 3/06; A61B 3/00; F21V 3/02; F21V 9/40

(52) U.S. Cl. CPC A61B 3/063; A61B 3/0083; A61B 3/0033; A61B 3/0008; F21V 3/02; F21V 9/40; A61B 2560/02; A61B 2560/04; F21W 2131/20

(57) ABSTRACT

A method for performing a full-field stimulus threshold (FST) test on a test subject, the method comprising: delivering at least one visual stimulus to at least one eye of the test subject, wherein the least one visual stimulus comprises a flash of light having an intensity and a duration, wherein the intensity of the flash of light is adjusted over the duration of the flash of light; obtaining at least one response from the test subject, wherein the at least one response corresponds to whether the test subject perceived the at least one visual stimulus; and determining the lowest intensity of light that the test subject is able to perceive based on the at least one response from the test subject.

### REFERENCE TO PENDING PRIOR PATENT APPLICATION

This patent application claims benefit of pending prior U.S. Provisional Patent Application Ser. No. 63/116,336, filed Nov. 20, 2020 by Diagnosys LLC and Jeffrey D. Farmer et al. for ENHANCED FST TECHNIQUES (Attorney's Docket No. DIAGNOSYS-11 PROV).

The above-identified patent application is hereby incorporated herein by reference.

### FIELD OF THE INVENTION

This invention relates to ophthalmic psychophysical diagnostic equipment in general, and more particularly to novel methods and apparatus for performing Full-field Stimulus Threshold (FST) testing.

### BACKGROUND OF THE INVENTION

Ophthalmic electrophysiology diagnostic equipment, such as that manufactured and sold by Diagnosys LLC of Lowell, MA, is typically used to stimulate the eye of a test subject using flashes (or moving patterns) of light, and to measure the resulting electrical response from the retina of the test subject (to obtain an electroretinogram, ERG) or from the visual cortex of the test subject (to obtain a visual evoked potential, VEP). Ophthalmic psychophysical diagnostic equipment stimulates the eye (or eyes) of the test subject using flashes of light during the test and then the test subject responds by indicating whether or not they have seen the flash of light. The response from the subject can be given verbally (e.g., “yes” or “no” or “no response”) and entered into the system by an observer (e.g., the clinician), or the response may be entered into the system by the test subject registering a response via a common input device (e.g., a keyboard or a subject response box, sometimes also referred to as a “button box”).

One such psychophysical test commonly performed on a test subject is the Full-field Stimulus Threshold (FST) test. Other common definitions of the FST acronym used by those skilled in the art include “Full-field Sensitivity Testing”, “Full-field Stimulus Testing” or other similar names to refer to the same type of ophthalmic test. Specifically, in the case of an FST test, the test is typically performed using a system which produces an audible tone that may (or may not) be followed by a flash of light (i.e., the “test flash” or “test flash stimulus”). Typically, a flash of light is produced contemporaneously with the audible tone, however, in a few instances during the course of the test, no flash is produced. This allows the clinician to check for a “false positive” response from the test subject (i.e., to control for the instance in which the test subject responds to the audible tone stimulus rather than the flash stimulus). After exposure to the audible tone stimulus (and, if applicable, the flash stimulus), the test subject then decides whether they saw (i.e., perceived) the flash of light, and the test subject inputs a corresponding “yes” or “no” into the system (e.g., using a button box or other input device). Then the test stimulus is repeated, i.e., another flash/tone combination is presented to the test subject. Although less commonly used, it is possible to perform an FST test that omits the audible sound (i.e., tone) stimulus that typically accompanies the flash stimulus. In such a modified FST test, the test subject is only required to provide a “yes” response when they actually perceive a test flash of light (and the test subject is not required to provide a “no” response when a flash of light is not perceived).

With FST tests such as those discussed above, the test flash stimulus is typically 1 millisecond or 200 milliseconds in length, however, if desired, the test flash stimulus can be shorter or longer. The system cycles through various flash luminance levels, in each case evoking a response from the subject with a goal of finding the flash level that is the lowest level of light the test subject can see, which is often referred to by those skilled in the art as the “threshold”. More particularly, the threshold is typically defined as the flash luminance level to which a subject responds “yes” 50% of the time and “no” 50% of the time, but other rules can be used to define the threshold, such as the minimum luminance level a subject could see 100% of the time during a test, etc. Various ways to calculate the threshold value from the test subject responses have been used and are described in the literature, including approaches that vary the order in which flashes of different luminance levels are presented to the test subject. One of the most common techniques comprises presenting the flashes to the test subject in a bracketed, random organization, and fitting the total set of subject responses during a test to a Weibull, or other similar mathematical function, in order to calculate the threshold. The test may be conducted with the test subject's pupils dilated or undilated.

Other common attributes of a typical FST test include:
(1) a set maximum test time;
(2) automatically measured dark adaptation time prior to the test;
(3) the number of reversals before finishing the test (a reversal is a test subject “yes” response followed by a test subject “no” response in sequence, or vice versa);
(4) display of the light luminance levels in dB, cd/m², and/or cd*s/m²;
(5) how much time the subject has to respond after presentation of the light/audible sound;
(6) the starting flash intensity used to begin the test;
(7) attributes of a fixation point (if used); and
(8) what audible sounds are used during the test, and other basic attributes for how the test is conducted and reported.

Aspects of how the test is conducted and reported help to judge the quality of the test taken by the test subject. During the test very high luminance flashes that the subject should see (based on their prior responses) are presented (i.e., to evaluate “false negative” responses), and in other cases no flash is presented at the time of an audible tone (evaluating ‘false positive’ responses). The overall quality of the FST test can further be assessed, either manually by a human (e.g., a clinician) or automatically using appropriate software, by various methods of analyzing the consistency of the test subject's responses across the whole test. These test quality attributes are typically included in the FST test report along with the threshold measured and other information from the test.

FIG. 1 is a schematic view showing an exemplary system 5 which may be used to perform FST tests on a test subject. System 5 generally comprises a stimulator 10 (e.g., a ganzfeld bowl stimulator, flash paddle or other similar device that produces light), a subject response box 15, a controller 20, a camera 25, and a computer 30 (with appropriate software). In use, stimulator 10 is configured to deliver a visual stimulus (e.g., a flash) to the eye(s) of a test subject, and the test subject registers whether the test subject perceived the visual stimulus using subject response box 15. Controller 20 is configured to control stimulator 10, and to convey test subject responses entered using controller 20 to computer 30. Appropriate software running on computer 30 is preferably used to analyze the responses and/or to modify the visual stimulus delivered to the test subject. Various physical setups are possible for system 5 and will be apparent to those skilled in the art in view of the present disclosure. However, the essential feature of prior art FST tests is that the light stimulator is positioned directly in front of both eyes of the subject and stimulates the entire visual field of the test subject's vision (i.e., the stimulation is “full field”). More specifically, testing using prior art systems has always been performed by presenting 1 flash (which may be 1 millisecond in duration or longer), at one time (sometimes referred to as a “test flash” or “test flash stimulus”) to which the subject responds using subject response box 15 to indicate whether or not they have seen the flash. The test flash, regardless of flash length, has always been a sharp square wave shaped flash. See FIG. 7. Also, prior art testing has generally been performed in either dark-adapted conditions (i.e., no background light on in the stimulator and no light on in the room, other than the flash stimulus) using various colors of flash (e.g., red, blue, green or white) or, in the few cases where light-adapted testing was performed, it was performed sub-optimally with separate controllers for the background light and test flash, and with limited background luminance levels and colors. And prior art FST testing has only been performed using the subject's full-field of vision. Finally, prior art FST testing has only been performed binocularly (i.e., testing both eyes at the same time using the same full-field stimulator 10) or monocularly (e.g., by covering one eye with an eyepatch). FST testing has not been performed using a dichoptic method (i.e., a method that stimulates each eye's field of vision independently at the same time).

FIGS. 2 and 3 show exemplary FST test reports 35 generated using an FST system such as system 5 discussed above. More particularly, FIG. 2 is a test report obtained by performing an FST test on a test subject having normal vision, wherein one test result is shown on the graph. The x-axis of the graph is a range of test flash luminance levels, which is typically plotted in a Log or dB scale, and the y-axis is the probability of detection of the flash stimulus by the test subject, in %. Test subject responses are recorded by the system (e.g., after being input by the test subject using subject response box 15) and plotted based on the test subject responses to the flashes during a test. Probability values of 0% shown in FIGS. 2 and 3 represent flash luminance level(s) to which the test subject always responded “no” (i.e., the test subject indicated that they did not perceive the flash stimulus). Probability values of 100% shown in FIGS. 2 and 3 represent flash luminance level(s) to which the test subject always responded “yes”. Probability values other than 0% and 100% (i.e., probability values falling within the range >0% to <100%) result when the test subject is presented with stimuli having the same flash luminance level multiple times, and the test subject responds “yes” some of the time and “no” some of the time. Computer 30 (i.e., software running on computer 30) automatically calculates the threshold (in this case defined at the 50% probability of detection), and records the threshold on the graph (FIGS. 2 and 3 show the threshold as indicated by a rectangle near the center of the graph at 40). Preferably, the software running on computer 30 also automatically presents the threshold value in a table.

FIG. 3 shows a test report 35 that is similar to the test report depicted in FIG. 2, however, FIG. 3 depicts a test report 35 resulting from a subject having low vision, and FIG. 3 also demonstrates that multiple test results may be plotted on the same graph.

FIGS. 4 and 5 show additional commercial examples of FST systems 45 that are generally similar to the exemplary system 5 discussed above, including the computer/software, controller, stimulator (with camera), and subject response box.

FIG. 6 shows three exemplary subject response boxes 15A, 15B and 15C. A two-button subject response box 15A is most commonly used, with one button of subject response box 15A being used to register a “yes” response, and a second button being used to register a “no” response, thereby creating a forced choice test. Response box 15B shows a more ergonomic version of two-button subject response box 15A. Alternatively, a single button response box 15C can be used instead of the two-button subject response boxes 15A and 15B, however, a single button response box 15C is not commonly utilized for FST testing.

The prior art FST tests currently being performed are limited to a specific visual stimuli (e.g., one flash delivered at one luminance level), and there is typically no variation in the visual stimuli used for the FST test. Furthermore, the prior art FST tests currently being performed are limited to binocular and monocular testing of the eye of a test subject.

Thus it will be seen that there exists a need in the art for a new and improved FST test which permits greater control over, and selective variation of, the visual stimuli used for the FST test, and which permits performing an FST test dichoptically.

### SUMMARY OF THE INVENTION

The present invention comprises the provision and use of novel methods and apparatus for performing enhanced FST testing, including permitting greater control over, and selective variation of, the visual stimuli used in the FST test, and which is configured to allow an FST test to be performed dichoptically.

In one preferred form of the invention, there is provided a method for performing a full-field stimulus threshold (FST) test on a test subject, the method comprising:
delivering at least one visual stimulus to at least one eye of the test subject, wherein the least one visual stimulus comprises a flash of light having an intensity and a duration, wherein the intensity of the flash of light is adjusted over the duration of the flash of light;
obtaining at least one response from the test subject, wherein the at least one response corresponds to whether the test subject perceived the at least one visual stimulus; and
determining the lowest intensity of light that the test subject is able to perceive based on the at least one response from the test subject.

In another preferred form of the invention, there is provided a method for performing a full-field stimulus threshold (FST) test on a test subject, the method comprising:
delivering a visual stimulus to at least one eye of the test subject, wherein the visual stimulus comprises a plurality of flashes of light, wherein each of the plurality of flashes of light has an intensity and a duration;
obtaining at least one response from the test subject, wherein the at least one response corresponds to whether the test subject perceived at least one of the plurality of flashes of light; and
determining the lowest intensity of light that the test subject is able to perceive based on the at least one response from the test subject.

In another preferred form of the invention, there is provided a method for performing a full-field stimulus threshold (FST) test on a test subject, the method comprising:
delivering at least one visual stimulus to at least one eye of the test subject, wherein the at least one visual stimulus comprises a flash of light having an intensity and a duration; wherein the at least one visual stimulus is delivered with background light, and further wherein a single controller is used to control the delivery of the at least one visual stimulus and the delivery of the background light;
obtaining at least one response from the test subject, wherein the at least one response corresponds to whether the test subject perceived the at least one visual stimulus; and
determining the lowest intensity of light that the test subject is able to perceive based on the at least one response from the test subject.

In another preferred form of the invention, there is provided a method for performing a full-field stimulus threshold (FST) test on a test subject, the method comprising:
delivering a first visual stimulus to a first eye of the test subject and a second visual stimulus to a second eye of the test subject, wherein the first visual stimulus and the second visual stimulus are provided by different stimulators;
obtaining at least one response from the test subject, wherein the at least one response corresponds to whether the test subject perceived the first visual stimulus or the second visual stimulus; and
determining the lowest intensity of light that the test subject is able to perceive, for at least one eye, based on the at least one response from the test subject.

In another preferred form of the invention, there is provided a method for performing a full-field stimulus threshold (FST) test on a test subject, the method comprising:
delivering at least one visual stimulus to at least one eye of the test subject, wherein the at least one visual stimulus is delivered to less than the full field of vision;
obtaining at least one response from the test subject, wherein the at least one response corresponds to whether the test subject perceived the at least one visual stimulus; and
determining the lowest intensity of light that the test subject is able to perceive based on the at least one response from the test subject.

In another preferred form of the invention, there is provided a method for performing a full-field stimulus threshold (FST) test on a test subject, the method comprising:
delivering a visual stimulus to at least one eye of the test subject, wherein the visual stimulus comprises a plurality of flashes of light, wherein each of the plurality of flashes of light has an intensity and a duration, wherein the intensity varies between each of the flashes of light, and further wherein the difference in the intensity between two flashes of light is different from the difference in the intensity between two other flashes of light;
obtaining at least one response from the test subject, wherein the at least one response corresponds to whether the test subject perceived the at least one visual stimulus; and
determining the lowest intensity of light that the test subject is able to perceive based on the at least one response from the test subject.

In another preferred form of the invention, there is provided a method for performing a full-field stimulus threshold (FST) test on a test subject, the method comprising:
delivering a visual stimulus to at least one eye of the test subject, wherein the visual stimulus comprises a plurality of flashes of light, wherein each of the plurality of flashes of light has an intensity and a duration, wherein the duration of time varies between each of the flashes of light, and further wherein the difference in the duration of time between two flashes of light is different from the difference in the duration of time between two other flashes of light;
obtaining at least one response from the test subject, wherein the at least one response corresponds to whether the test subject perceived the at least one visual stimulus; and
determining the lowest intensity of light that the test subject is able to perceive based on the at least one response from the test subject.

In another preferred form of the invention, there is provided apparatus for performing a full-field stimulus threshold (FST) test on a test subject, the apparatus comprising:
a stimulator for delivering at least one visual stimulus to at least one eye of the test subject, wherein the at least one visual stimulus comprises at least one flash of light, wherein the at least one flash of light has an intensity, a duration and a color;
a controller for controlling the intensity, the duration and the color of the at least one flash of light;
a test subject input device for obtaining at least one response from the test subject, wherein the at least one response corresponds to whether the test subject perceived the at least one visual stimulus; and
a processor for determining the lowest intensity of light that the test subject is able to perceive based on the at least one response from the test subject.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic view of an exemplary prior art system for performing an FST test on a test subject;

FIGS. 2 and 3 show exemplary test reports for FST tests performed on different test subjects;

FIGS. 4 and 5 are schematic views of exemplary systems for performing an FST test on a test subject;

FIG. 6 is a schematic view showing three exemplary subject response input devices;

FIG. 7 is a graph illustrating a square wave flash shape used in prior art FST testing systems;

FIGS. 8A-8E show novel wave flash shapes formed in accordance with the present invention which may be used in FST testing systems;

FIG. 9 is a graph illustrating a test subject FST score obtained using a traditional 4 millisecond flash stimulus;

FIG. 10 is a graph illustrating a test subject FST score obtained using a novel flicker flash stimulus formed in accordance with the present invention;

FIG. 11 is a graph illustrating a test subject FST score obtained using a novel flicker flash test stimulus formed in accordance with the present invention together with a novel 100 cd/m² 6500K white light background formed in accordance with the present invention;

FIG. 12 is a schematic view showing a novel system for performing a dichoptic FST test formed in accordance with the present invention; and

FIG. 13 is a schematic view showing a novel stimulator for performing a partial field FST test formed in accordance with the present invention.

### DETAILED DESCRIPTION OF THE PREFERRED EMBODIMENTS

The present invention comprises the provision and use of novel methods and apparatus for performing enhanced FST testing, including permitting greater control over variation of the visual stimuli used in the FST test. The present invention also comprises the provision and use of new methods and apparatus to perform an FST test dichoptically.

More particularly, the present invention generally comprises seven new methods and/or apparatus for FST testing that can be used independently of one another, or in combination with one another. The new methods and apparatus of the present invention extend and improve the FST test (and hence, deliver a superior clinical result). A first novel method according to the present invention comprises presenting new test flash shapes to the test subject. A second novel method according to the present invention utilizes more than one flash (i.e., a “flicker”) as the visual stimulus that is presented to the test subject. A third novel method according to the present invention comprises the provision and use of a background light for light-adapted FST tests, wherein the background light comprises a wide range of highly-controlled luminance levels and colors, which background light is controlled by the same controller as the test flash (i.e., the visual stimulus), and which uses the same stimulator as that used to deliver the test flash to the test subject. A fourth novel method according to the present invention comprises a novel dichoptic system for performing an FST test on a test subject. A fifth novel method according to the present invention comprises performing an FST test on a test subject while utilizing less than the full-field of vision, i.e., testing the partial field of one or both eyes at a time. A sixth novel method according to the present invention comprises varying the luminance step value allowed between flashes. A seventh novel method according to the present invention comprises varying the amount of time between test flashes.

### 1. Test Flash Shape

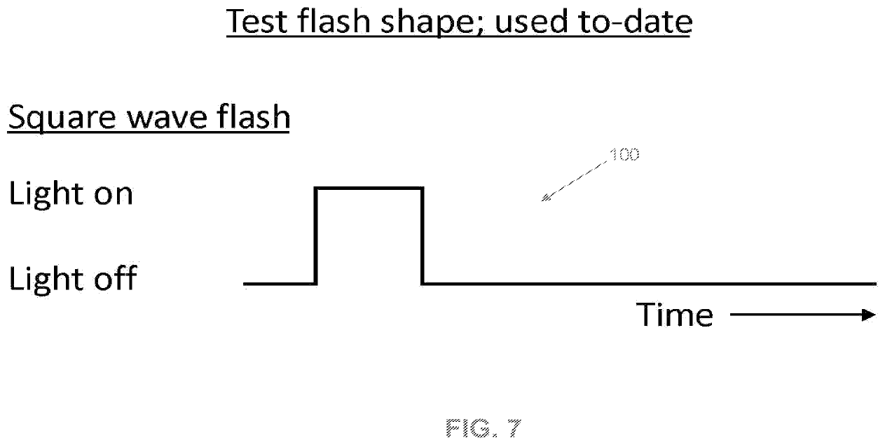

Historically, and looking now at FIG. 7, only square wave shaped flashes 100 have been used for FST test flashes. With a “square shaped” flash 100, there is a sharp “on” and sharp “off” as the light of the visual stimulus (i.e., the flash) is presented to the test subject. Both the length of the flash (indicated by the x-axis) and the luminance level (indicated by the y-axis) are arbitrary.

With the novel methods of the present invention, new flash shapes are provided, with each novel flash shape having its own characteristics and advantages. Given that the human eye and vision system react differently to light coming on, light going off, as well as light going on and off at different rates (e.g., sharp, ramp, gradual, etc.), there are advantages to the novel method of the present invention, which permits a wider array of flash types to be used for FST testing.

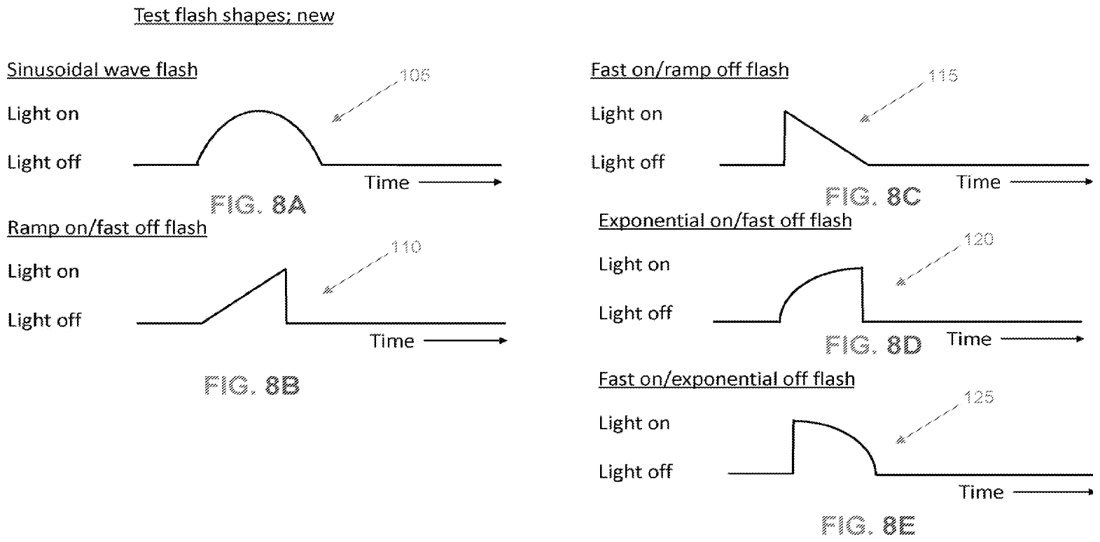

Looking next at FIGS. 8A-8E, there are shown various novel flash shapes formed in accordance with the present invention.

More particularly, FIG. 8A shows a sinusoidal wave flash 105 formed by varying the luminance level of the flash as a function of time, such that the flash reaches full luminance at a time approximately one-half of the total flash stimulus, and decays in luminance at the same rate until the flash stimulus is complete.

FIG. 8B shows a ramp on/fast off flash 110 formed by increasing the luminance level of the flash linearly as a function of time, and then turning off the light entirely once the flash stimulus is complete.

Fast on/ramp off flash 115 (FIG. 8C) is the inverse of ramp on/flash off flash 110. With fast on/ramp off flash 115, the luminance level of the flash stimulus is presented at its peak level essentially when the flash stimulus is first presented, and the luminance level of the flash stimulus then decays linearly as a function of time over the course of the flash.

FIG. 8D shows an exponential on/fast off flash 120 formed by increasing the luminance level of the flash exponentially as a function of time, and then turning off the light entirely once the flash stimulus is complete.

Fast on/exponential off flash 125 (FIG. 8E) is the inverse of exponential on/fast off flash 120. With fast on/exponential off flash 125, the luminance level of the flash stimulus is presented at its peak level essentially when the flash stimulus is first presented, and the luminance level of the flash stimulus then decays exponentially as a function of time.

Other flash shapes will be apparent to those of ordinary skill in the art in view of the present disclosure.

It will thus be appreciated that the present invention comprises the provision and use of the new flash types utilizing various strategies employed for when the light is turned on and turned off. Each of the new test flash shapes of the present invention can be utilized over an arbitrary length of flash time, as well as for a single flash or a flicker flash, using the full range of flash and background color and luminance levels.

### 2. Stimuli Using Multiple Flashes

As discussed above, with prior art FST tests, a test flash is typically presented to a subject as a single flash (accompanied by an audible tone); the test subject is instructed to respond to the audible tone and indicate whether or not they saw (i.e., perceived) the flash (e.g., with a “yes” or “no” response). Single flashes of various durations (i.e., flash lengths) have been used in prior art FST tests, with flashes having durations typically ranging from 1 millisecond up to 1 second, and with the most common flash duration being 4 milliseconds or 200 milliseconds. With prior art visual electrophysiology (ERG) tests, flashes having durations of 4 milliseconds are typically utilized due to certain ERG-specific test benefits realized in ERG tests of that flash length. Use of flashes having 4 millisecond durations has generated interest in using that same flash length in FST psychophysical testing. A flash having a duration of 200 milliseconds is also a common flash length that is used in other prior art psychophysical tests such as Visual Field automated perimetry, and therefore flashes having a duration of 200 milliseconds has been a logical choice for use in FST.

However, there are important tradeoffs between using flashes that are 4 milliseconds in duration versus using flashes that are 200 milliseconds in duration when performing FST tests. By way of example but not limitation, one benefit of using a 4 millisecond flash for FST testing is that the flash is faster (i.e., faster than the much longer 200 millisecond flash duration), giving the test subject more time after the flash to determine if they saw the flash and to enter their response (within a testing period having a fixed amount of time between flashes). Furthermore, the human eye responds best to contrast in light, so positioning the “light on” very near to the “light off”, which in a 4 millisecond flash is only 4 milliseconds apart, makes it easier for the human eye and brain to detect the presence of a flash.

By way of further example but not limitation, one benefit of using a 200 millisecond flash for FST testing includes the fact that test subjects ordinarily continue to blink naturally during a test, and by using a longer test flash it is more likely that the test subject will not completely miss the flash (i.e., because the flash stimulus is by chance initiated at the same moment that the test subject blinks) even in the presence of ordinary blinking.

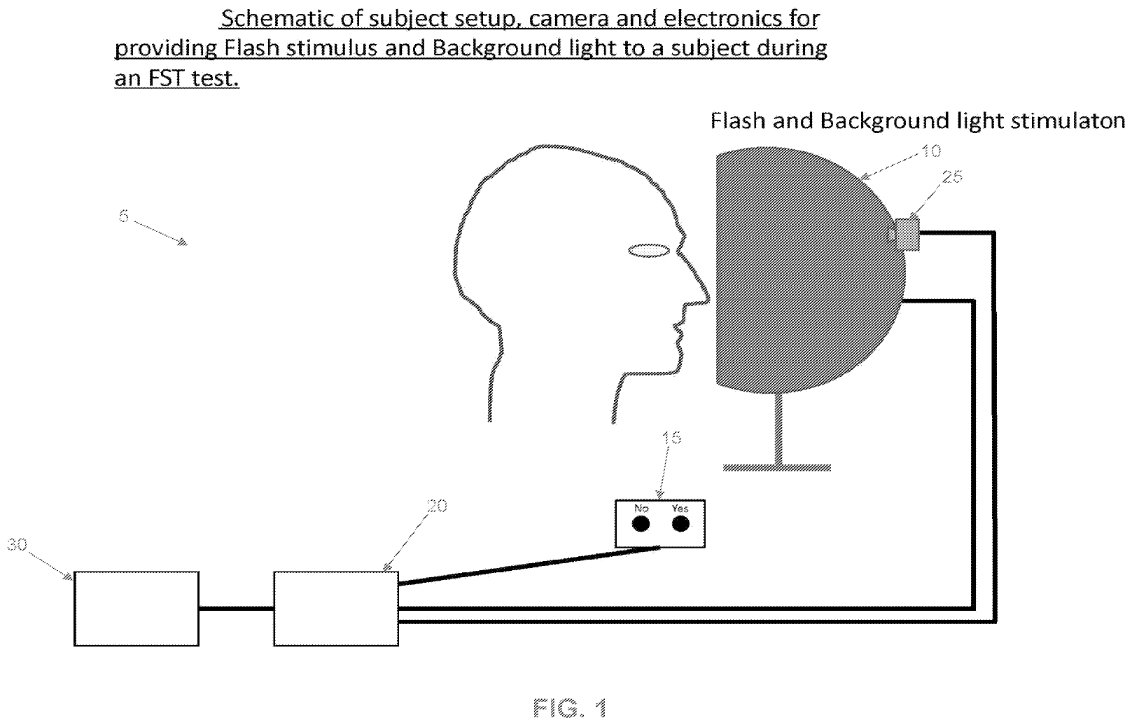

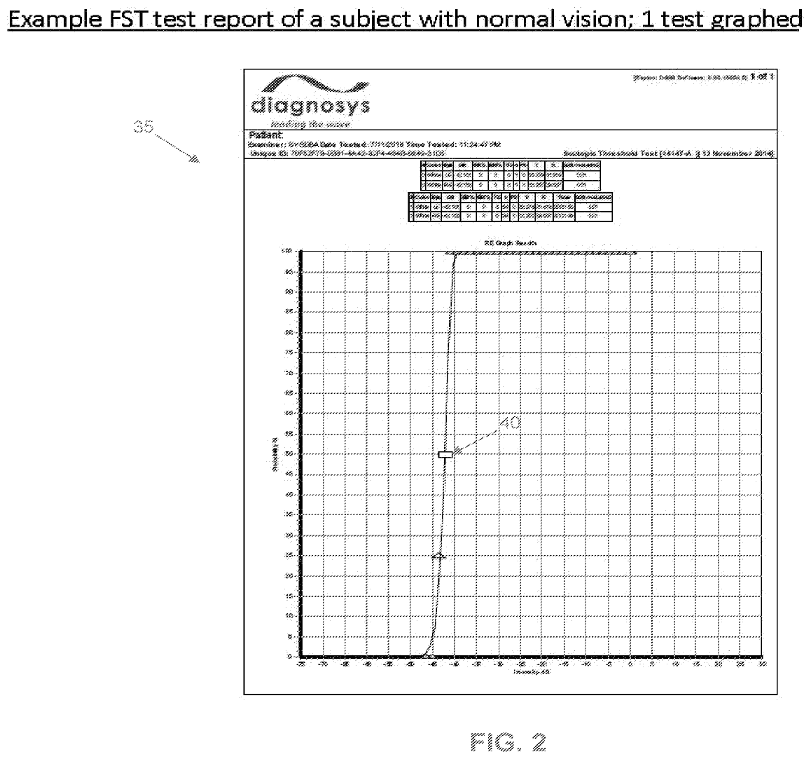

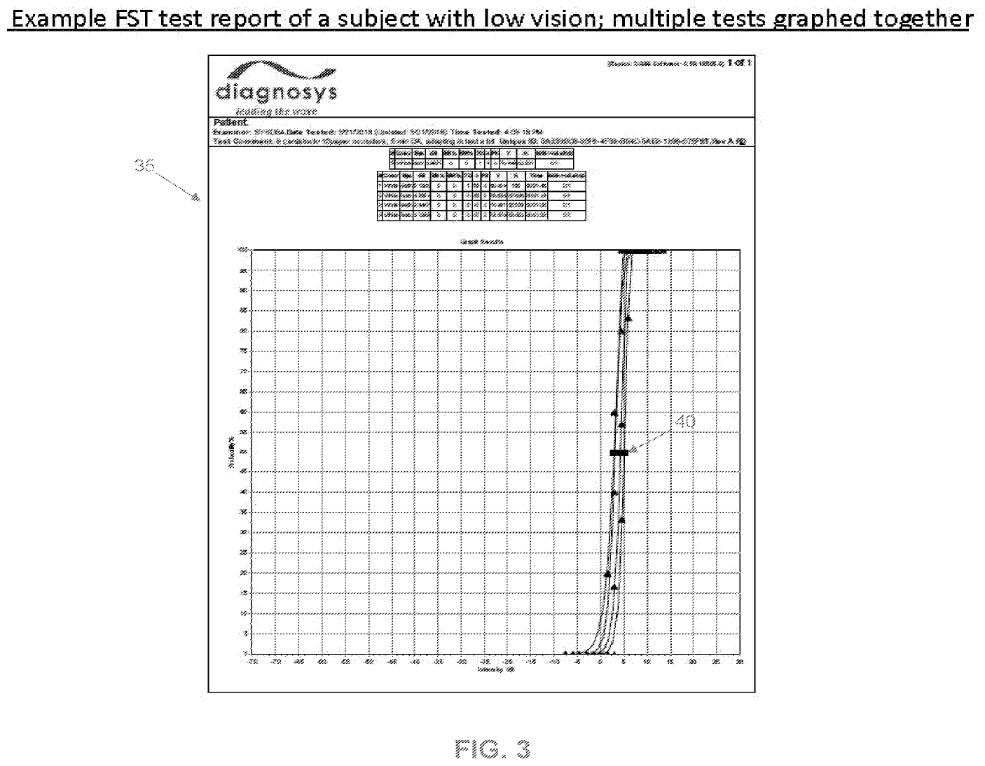

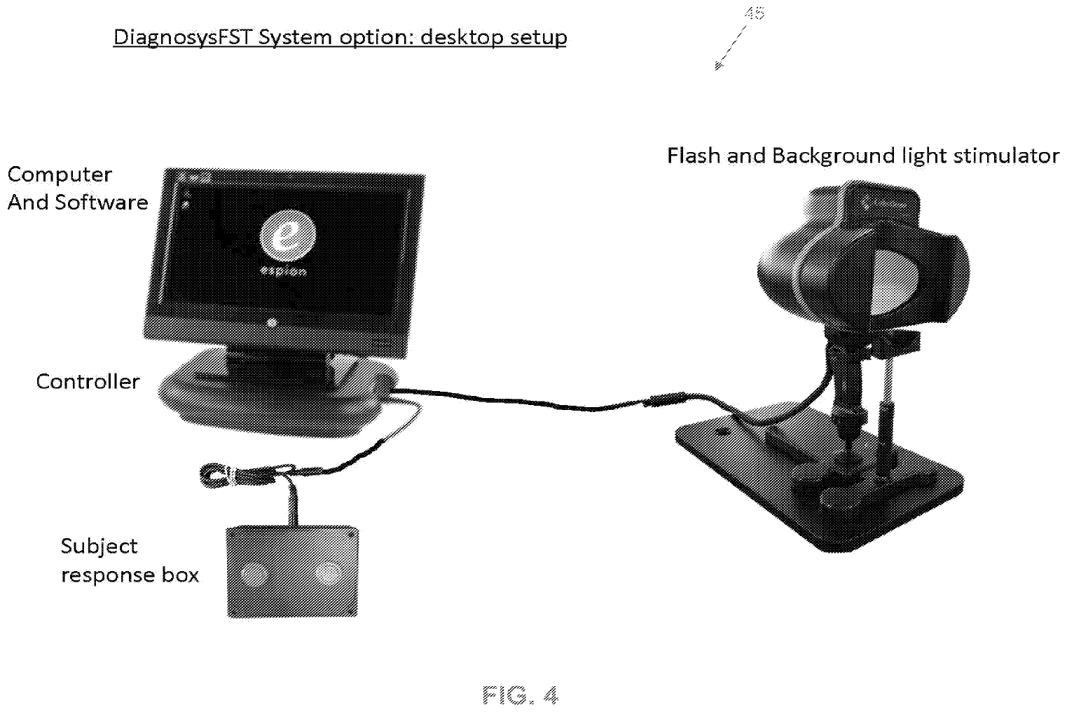

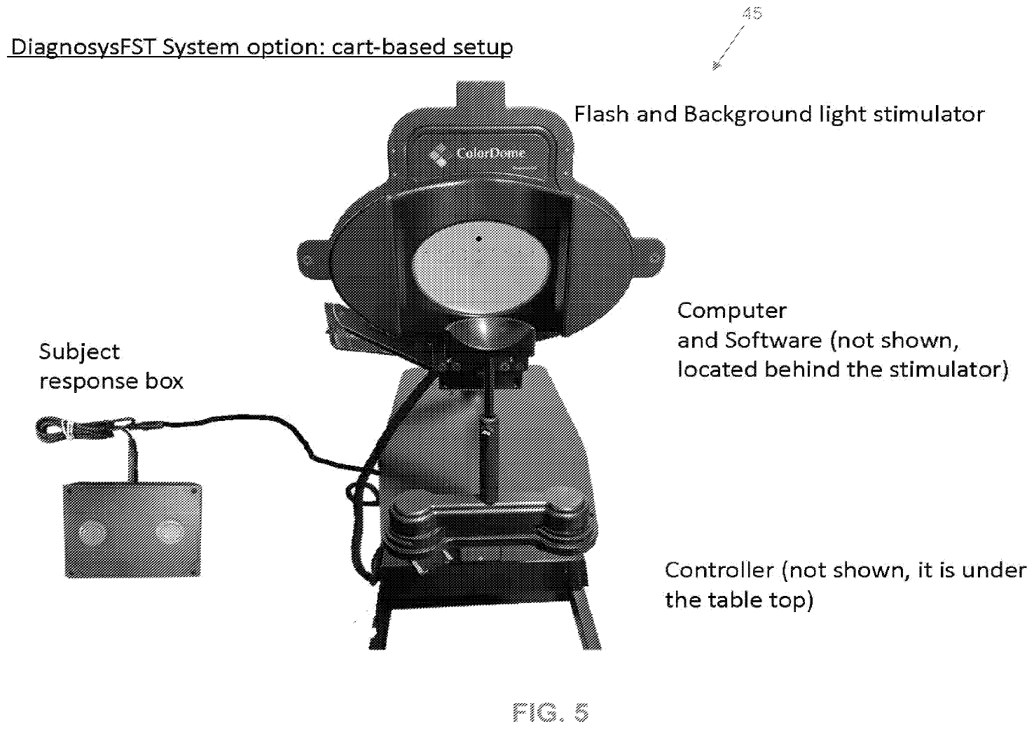

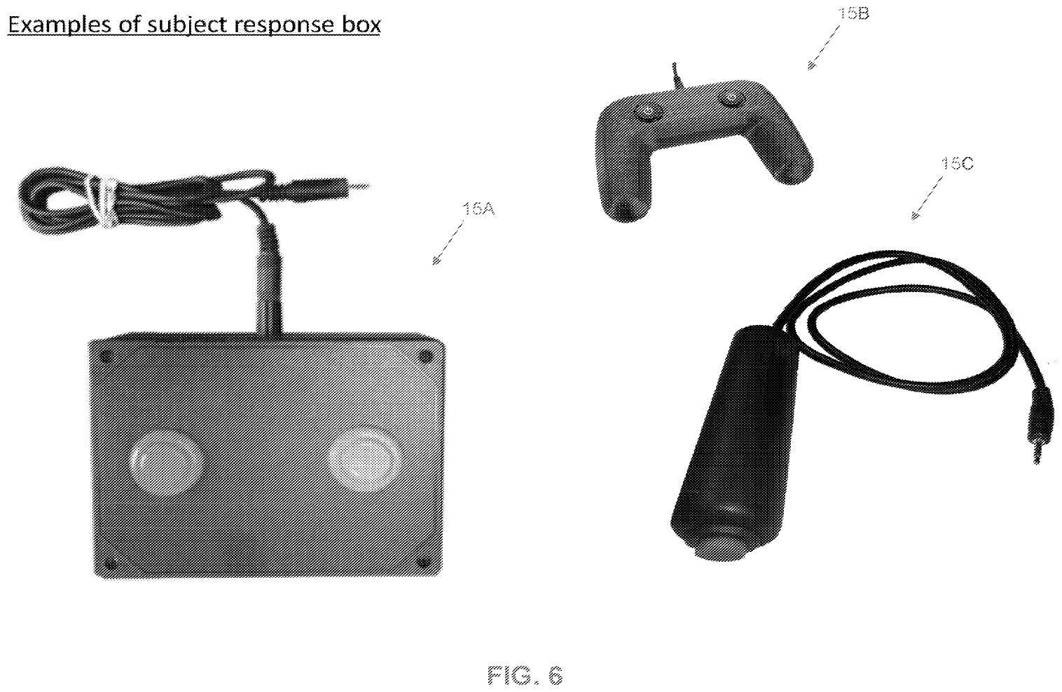

The present invention comprises the use of a novel approach that combines the best attributes of the aforementioned two test flash duration strategies (i.e., 4 millisecond and 200 millisecond) a flicker test flash stimulus. A flicker test flash stimulus is defined as a set of more than one flash, typically at a fixed frequency between each flash, delivered to the eye(s) of the test subject over a short period of time. By way of example but not limitation, one preferred embodiment of the flicker test flash stimulus of the present invention delivers 4 millisecond flashes flashing at a rate of 10 Hertz (i.e., frequency) for a period of 200 milliseconds. When implemented automatically in software (e.g., via computer 30 of the aforementioned exemplary FST system shown in FIG. 1), the present invention can enable three 4 millisecond flashes to be delivered to the eye(s) of the test subject during the period of 200 milliseconds (i.e., a first flash at time 0, a second flash at time 100 milliseconds and a third flash at time 200 milliseconds).

The flicker test flash approach of the present invention benefits from both the short (e.g., 4 milliseconds) and the long (e.g., 200 milliseconds) flash durations typically used in the single flash approach. Specifically, fast individual flashes that are easier to detect and that occur over a longer period of time help lower the risk that a naturally-occurring blink occurs contemporaneously with the test flash. With the flicker test flash stimulus of the present invention, the “test flash” is a set of three, highly controlled flashes that are emitted in a rapid sequence (i.e., so as to appear as a flicker). The test subject is then asked (i.e., immediately after the flicker flash stimulus has been delivered) to respond to whether or not they saw (i.e., perceived) the flicker test flash (i.e., “yes” or “no”). Alternatively and/or additionally, the test subject may be asked whether they perceived all of, or even just a portion of, the flicker test flash stimulus.

If desired, other flicker test flash stimulus options may be used, including flicker test flash stimuli comprising frequencies that range from 1 to 120 hertz, and/or flicker test flash stimuli comprising individual flash durations ranging from 0.25 milliseconds up to 30 milliseconds. And, if desired, the duration of the combined flashes that collectively make up the flicker test flash stimulus can range from 4 milliseconds up to 2 seconds. It should be appreciated that, if desired, the frequencies of the flicker test flash stimuli may be higher or lower, and/or the duration of each individual flash stimuli may be shorter or longer. Furthermore, it should also be appreciated that, if desired, the flicker test flash stimuli may incorporate a variable frequency, and/or variable individual flash duration. And, if desired, flicker test flash stimuli may comprise an arbitrarily-selected color (e.g., white, red, blue, green, etc.), differing colors during the presentation of the stimulus, and/or presenting the stimulus either with no background light or with a background light. It should also be appreciated that, if desired, the flicker test flash stimuli may also be presented using the full array of flash shapes discussed above (e.g., to vary the luminance level of the flicker test flash stimulus during presentation of the stimulus to the eye(s) of the test subject.

Due to the benefits of using the flicker test flash stimulus of the present invention when performing an FST test (when compared to the traditional single flash approaches discussed above), a test subject often able to score better (i.e., receive an FST test measurement that indicates a lower threshold of light that can be perceived by the patient). This test result is likely more indicative of the test subject's true low light threshold.

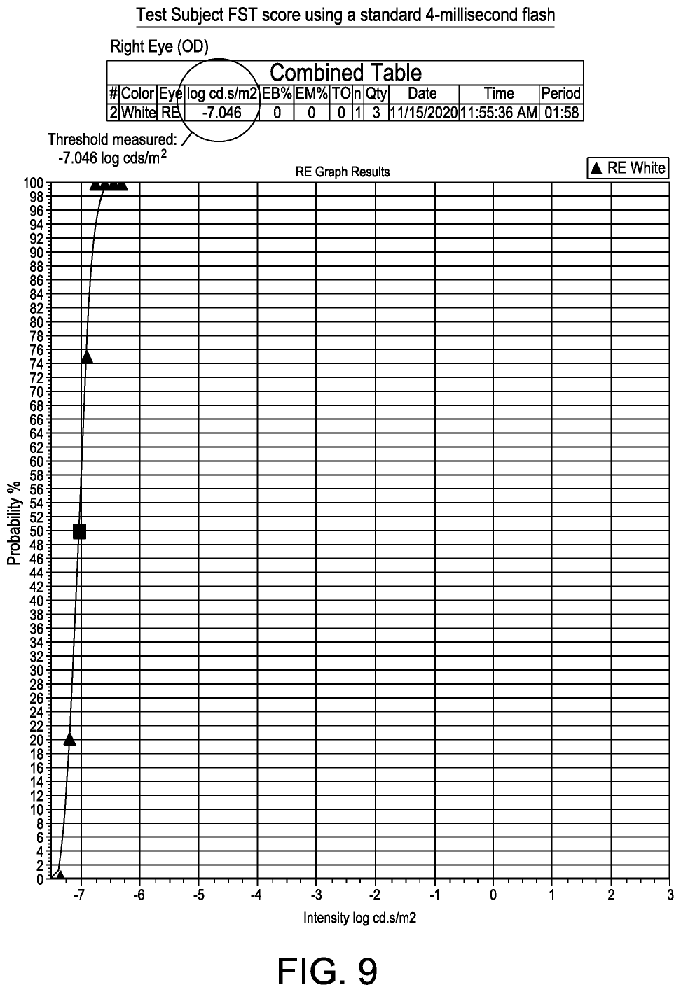

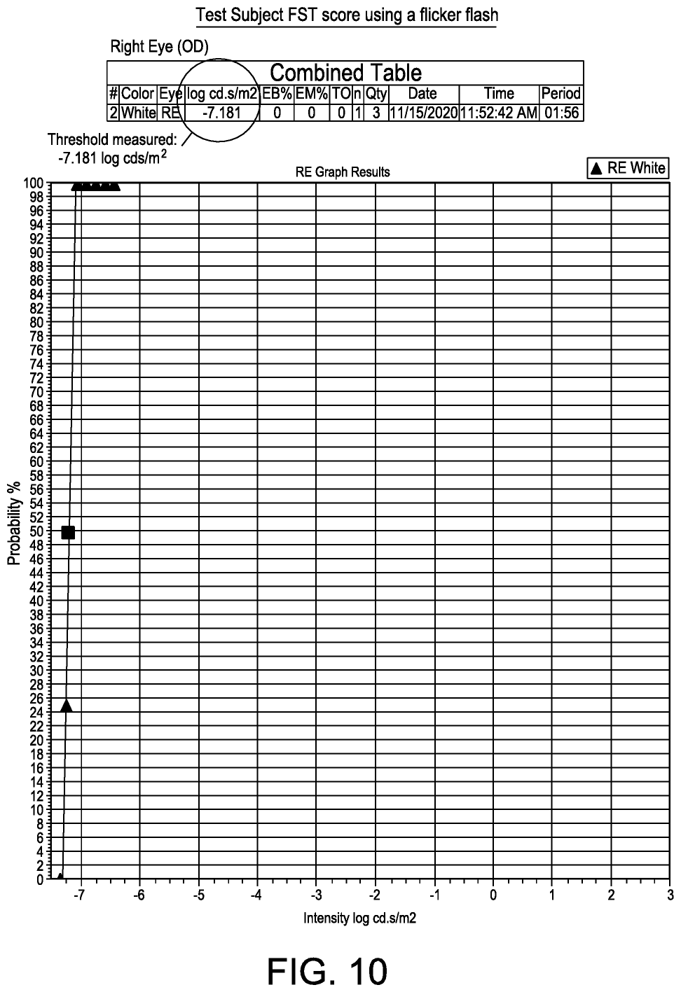

See, for example, FIGS. 9 and 10, which show the results of an FST test in which a test subject was tested using a dark-adapted white flash FST using a prior art single-flash 4 millisecond flash technique (FIG. 9) and a flicker test flash stimulus formed in accordance with the present invention (FIG. 10). In this case the threshold measured using the prior art single flash is −7.046 Log cd*s/m², and the threshold measured using the flicker test flash stimulus of the present invention is −7.181 Log cd*s/m². Converting these Log values into cd*s/m² units gives: 8.99×10⁻⁸ cd*s/m² and 6.59×10⁻⁸ cd*s/m², showing a 36% improvement in lower threshold measured for the subject using the flicker test flash stimulus of the present invention, demonstrating a strong improvement in the FST test through use of the novel flicker test flash technique of the present invention.

### 3. Light-Adapted FST

Prior-art based FST testing has either been done in dark-adapted conditions (i.e., test conditions in which there is no background light on in the stimulator and there is no light on in the room during the test except for the test flash stimulus) using flashes of various colors (e.g., red, blue, green or white) or, in the few cases where light-adapted testing was performed, testing was performed sub-optimally with separate controllers for the background light (if a controller was used at all for the background light) and the test flash, and with limited background luminance levels and colors. The present invention enables test flashes (which may be a single short flash, a long flash, or a flicker test flash stimulus of the type discussed above) of arbitrary color/luminance level/shape, and a background light of arbitrary color/luminance level to be presented to the test subject using one common controller and computer. The benefits of using one common controller to control both the flash stimulus and background light include the ability to synchronize or modulate the two variables (i.e., the flash stimulus and the background light) as well as to ensure that both are well calibrated light for both luminance and color, and to enable a more cost effective and space efficient clinical system that is easy to use by medical clinicians.

Furthermore, the present invention extends beyond the prior art in important ways relating to both luminance levels of background light and color of background light. Previously, with prior art FST testing systems, only approximately 100 cd/m 2 background light was provided by the stimulator (e.g., the aforementioned exemplary stimulator 10) due to technical limitations, using a controller separate from the controller which controlled the flash stimulus. The present invention can control very dim background lights all the way down to the threshold of a subject's perception whereas prior art systems were unable to do so.

The present invention comprises the provision and use of background light to be presented during the FST test that extends up to and over 10,000 cd/m², background light which can be presented in various (i.e., arbitrary colors), and background light which is controlled using the same controller that is used to control the test flash stimuli. The present invention also comprises the provision and use of background luminance levels which include, but are not limited to, 30 cd/m², 100 cd/m², 1000 cd/m², and up into the 10,000's of cd/m², in a variety of white colors (e.g., 2700K, 3500K, 6500K, etc.) as well as other colors including, but not limited to, red, blue, green, and combinations (i.e., ordered or arbitrary) of the individual colors to create other mixes of background light. Additionally, with the present invention, the background light color used in an FST test may be the same, or different from, the color used for the test flash and, if desired, the background light may be changed during the test.

Stimulus light-adapted FST tests are typically designed so as to enable the testing of cone photoreceptor pathways in the human eye and visual system. High background light luminance levels are required during the FST test in order to ensure that no rod photoreceptor pathways in the human eye are contributing to the test subject's ability to see light. It will be appreciated that inasmuch as the present invention comprises the ability to control the background light using the same controller as the controller used for controlling the test flash stimuli, and inasmuch as the present invention facilitates the use of background light having luminance levels of 100 cd/m² or greater, the present invention is a significant advance over prior art FST tests for testing cone photoreceptor pathways in the human eye and visual system.

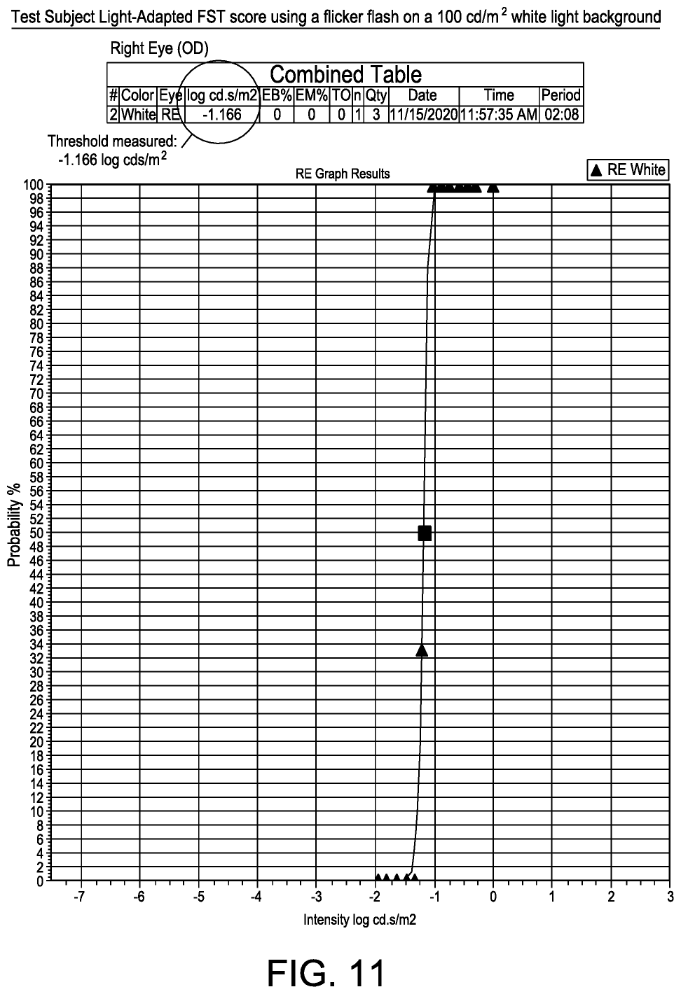

Thus it will be seen that the novel approach of the present invention may be used for research, clinical trials, and for clinical use of FST when the test subject's rod and cone pathway-driven FST thresholds need to be determined definitively and separately. FIG. 11 shows an exemplary test report for a test subject's light-adapted FST test according to the present invention, in which a white light flicker test flash stimulus and 100 cd/m² 6500K white light background was used during the FST test to measure the subject's cone pathway driven threshold to white light test flashes. In this case, a threshold of −1.166 Log cd*s/m² was measured during the test.

### 4. Dichoptic FST

Prior art FST testing has been conducted either binocularly or monocularly on the eye(s) of a test subject. In either case, one stimulator is used to present test flash stimuli to either both eyes at the same time (i.e., binocular) or, with an eye patch over one of the test subject's eyes, to present test flash stimuli to one eye of the test subject at a time (i.e., monocular).

The present invention enables dichoptic FST testing. Dichoptic is defined as viewing a separate and independent field by each eye. In a dichoptic presentation, a first stimulus is presented to a first eye of the test subject (e.g., the left eye), and a second, different stimulus is presented to the other eye of the test subject (e.g., the right eye).

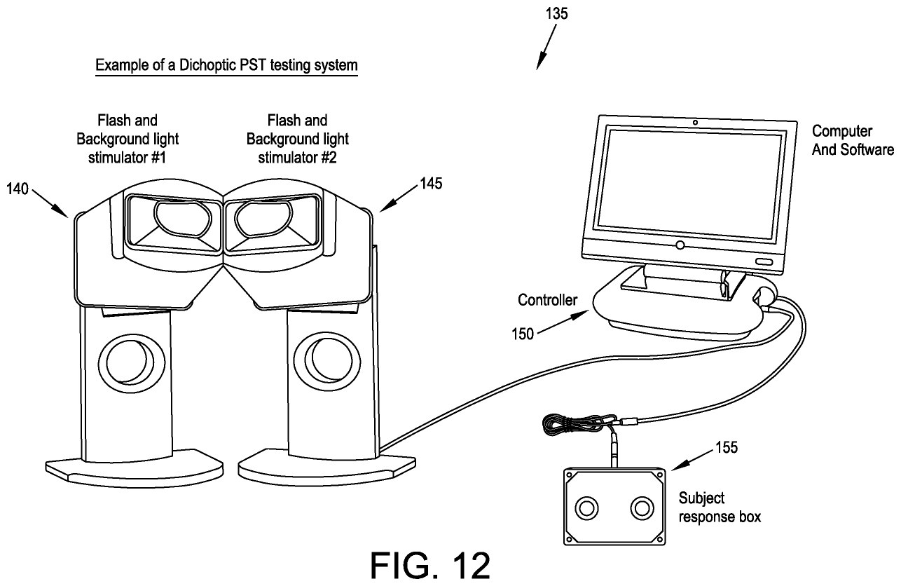

FIG. 12 is a schematic view showing the primary components used in a novel dichoptic FST system 135 formed in accordance with the present invention. More particularly, dichoptic FST system 135 generally comprises a first stimulator 140 configured to selectively deliver a visual stimulus to a first eye of a test subject, a second stimulator 145 configured to selectively deliver a visual stimulus to a second eye of a test subject, a controller 150 for controlling attributes of the visual stimuli delivered to the test subject's eye(s) by stimulators 140, 145, a subject response box 155 for registering the test subject's response to the visual stimulus, and a computer 160 comprising appropriate software for recording responses received by subject response box 155 and/or for controlling controller 150. It will be appreciated that dichoptic FST system 135 may comprise individual (i.e., physically separated components), as shown schematically in FIG. 12 or, if desired, dichoptic FST system 135 may be integrated into a single housing (not shown) and controlled by a single controller and computer.

While standard binocular and monocular FST tests can be performed using dichoptic FST system 135, there are numerous additional advantages to performing FST tests using a dichoptic FST system, as well as entirely new types of FST tests that are enabled by the present invention.

First, it will be appreciated that monocular testing becomes much more efficient for the test subject and the clinician conducting the test when using dichoptic FST system 135. For both light-adapted and dark-adapted testing using dichoptic FST system 135, there is no need to cover the non-tested eye with an eyepatch (i.e., because the fellow, i.e., untested, eye is not exposed to the visual stimulus). Dichoptic FST system 135 can present a dark environment or a light environment, as required, to the eye of the test subject which is not being tested (i.e., the eye of the test subject which is not exposed to the visual stimulus), while presenting the dark-adapted or light-adapted FST test to the other eye being tested (i.e., the eye of the test subject which is being exposed to the visual stimulus). Thus, the present invention reduces labor time and cost to conduct an FST test, ensures high consistency of light presentation to both the tested and non-tested eyes (i.e., because it is quite common for eyepatches used in prior art systems to not fully cover the non-tested eye and that issue is completely avoided with dichoptic FST system 135).

Binocular FST tests may also be conducted using dichoptic FST system 135, which may be configured to present exactly the same test flash and background to both stimulators at any given time.

Dichoptic FST system 135 also enables completely new types of tests that have either economic or medical benefits (or both) and which have never been performed before. By way of example but not limitation, dichoptic FST system 135 permits monocular FST tests to be completed in an automatic fashion, one eye after the other. Alternatively, if desired, the individual eye FST tests may be interleaved, i.e., each eye may be tested individually, however, the test flash stimuli may be configured to alternate in an arbitrary or methodical manner between the two eyes (i.e., first stimulator 140 may be used to stimulate the first eye of the test subject, and then second stimulator 145 may be used to stimulate the second eye of the test subject, etc.).

It will also be appreciated that dichoptic FST system 135 also allows different combinations of color, frequency and other parameters to be used in the FST test, and further, the different combinations of stimuli may be interleaved, for one eye, and also between the eyes on dichoptic FST system 135.

Additional new test types enabled by dichoptic FST system 135 of the present invention include, but are not limited to:
(1) utilizing different and/or varying controlled background lights of arbitrary color and luminance levels for the two eyes of the test subject during the test;
(2) utilizing different and/or varying controlled test flash stimuli of arbitrary color and luminance levels as well as flash type (e.g., single or flicker) and flash shape (e.g., one or more of the novel flash shapes discussed above) for the two eyes during the FST test, with the flash stimuli being delivered to the two eyes at the same time (or at different times); and

(3) numerous other combinations of flash stimuli and background light across the two stimulators 140, 145.

### 5. Partial Field FST

Prior art FST tests have always been performed by stimulating the test subject's eye(s) full field of view. Separately, automated perimetry systems exist which test a test subject's eye light thresholds and always use spot sizes that are 2 degrees of the field of view or smaller.

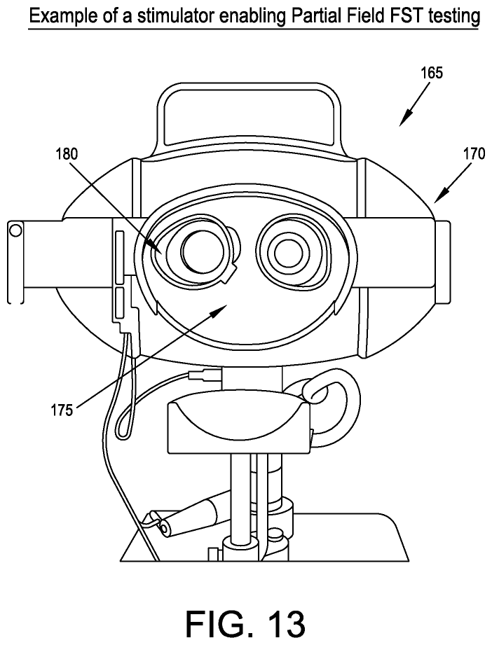

The present invention comprises a novel partial field FST system which enables testing of a test subject's eye light thresholds using spot sizes that are less than full-field and greater than 2 degrees of the field of view. More particularly, and looking now at FIG. 13, there is shown a novel partial field FST system 165 formed in accordance with the present invention. A device 170 may be mounted onto a full-field stimulator 175 of partial field FST system 165, whereby to provide a partial field view for each eye of the test subject into the stimulator 175 that provides flash stimuli and background light. Eye shields 180 may or may not be mounted to stimulator 175.

With partial field FST system 165 configured in the manner shown in FIG. 13, it is possible to test a field of view of the test subject. Partial field FST tests performed using partial field FST system 165 may utilize any of the FST techniques disclosed in the prior art, and/or partial field FST system 165 may utilize any of the novel aspects of the general FST test relating to test flash luminance/length/color, background luminance/color, flash shape, dichoptic presentation, etc., as disclosed herein. Partial field FST testing is important because most eye diseases do not affect the full field of site, and therefore a less than full-field test will frequently be more sensitive and specific to a given eye disease.

### 6. FST Using More than One Luminance Step Size

Prior art FST tests have always used a single, fixed step size (i.e., the minimum increment between any two possible flash luminance levels during the test) in order to determine the difference between luminance levels presented to a test subject. By way of example but not limitation, the difference between all luminance levels that may be used in one test may be in increments of 0.15 Log cd*s/m² or 0.20 Log cd*s/m² (or in various other units of measure such as Log cd/m² or 1.5 dB). However, there has never been a combination of two or more step size values utilized in a single FST test.

The present invention comprises the provision and use of more than one step size between luminance levels used in the same FST test. By way of example but not limitation, in one form of the present invention, larger steps sizes such as 0.5 Log cd*s/m² are utilized until an “approximate” threshold is determined, and then smaller step sizes such as 0.15 Log cd*s/m² are utilized in order to refine the threshold measured. It will be appreciated that there exist other combinations of step sizes that may utilize one or more luminance step sizes in a single FST test. Such “more than one luminance step size” values may be pre-determined using a set of rules for the test, or the luminance step size values may be dynamically determined during the test itself. Luminance step sizes may also be chosen during the test (e.g., by the clinician administering the test), and/or luminance step sizes may be changed based on prior information recorded during that test or from other tests previously recorded for that test subject (or for other test subjects). Additionally, and/or alternatively, the luminance of the very first flash stimulus presented may be determined based on information recorded during other tests previously recorded for that particular test subject (or for other test subjects). Each of these algorithmic approaches help improve the speed and accuracy of the FST test, thereby providing economic and medical benefits.

### 7. FST Using More than One Time Step Size

Prior art FST tests have always used a single, fixed amount of time between flash stimuli presented to a test subject. By way of example but not limitation, the time difference between all flash stimuli presented during a test may be 2 seconds or 3 seconds, however, historically, there never has been a combination of two or more time step sizes in a single FST test.

The present invention comprises the provision and use of more than one time interval step size between flash stimuli used in the same FST test. By way of example but not limitation, one technique of the present invention comprises using shorter interval time step sizes (e.g., 2 seconds) between flash stimuli until an approximate threshold is found, and then using longer time step sizes (e.g., 5 seconds) between flash stimuli to refine the threshold measured when more subject concentration is required.

Other combinations of the foregoing technique are encompassed by the present invention, wherein all such combinations use more than one time step size in one FST test.

Such “more than one time interval step size” values may be pre-determined using a set of rules for the test, or the time interval step size values may be dynamically determined during the test. Time step sizes may also be chosen during the test (e.g., by the clinician administering the test), and changed based on prior information recorded during that test or from other tests previously recorded for that particular test subject (or other test subjects). Each of these algorithmic approaches help improve the speed and accuracy of the FST test, thereby providing economic and medical benefits.

### Modifications of the Preferred Embodiments

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. A method for performing a full-field stimulus threshold (FST) test on a test subject, the method comprising:
delivering at least one visual stimulus to at least one eye of the test subject, wherein the least one visual stimulus comprises a flash of light having an intensity and a duration, wherein the intensity of the flash of light is adjusted over the duration of the flash of light;
obtaining at least one response from the test subject, wherein the at least one response corresponds to whether the test subject perceived the at least one visual stimulus; and
determining the lowest intensity of light that the test subject is able to perceive based on the at least one response from the test subject.

2. The method of claim 1 wherein the intensity of the flash of light is adjusted so that the flash of light reaches full intensity at a time approximately one half of the total duration of the flash of light and then decays until the flash of light is complete.

3. The method of claim 1 wherein the intensity of the flash of light is adjusted so that the intensity of the flash of light increases linearly during the duration of the flash of light and then is turned off entirely once the flash of light is complete.

4. The method of claim 1 wherein the intensity of the flash of light is adjusted so that the intensity of the flash of light is presented at the highest intensity and then decays linearly over the duration of the flash of light.

5. The method of claim 1 wherein the intensity level of the flash of light is adjusted so that the intensity of the flash of light increases exponentially over the duration of the flash of light and then is turned off entirely once the flash of light is complete.

6. The method of claim 1 wherein the intensity of the flash of light is adjusted so that the intensity of the flash of light is presented at the highest intensity and then decays exponentially over the duration of the flash of light.

7. The method of claim 1 wherein a plurality of flashes of light are delivered to the test subject.

8. The method of claim 1 wherein the at least one visual stimulus is delivered to both eyes of the test subject.

9. The method of claim 8 wherein the at least one visual stimulus is delivered simultaneously to both eyes of the test subject, with one eye receiving a stimulus of a first intensity and first duration and the second eye receiving a stimulus of a second intensity and second duration.

10. The method of claim 1 wherein the at least one visual stimulus is delivered with no background light.

11. The method of claim 1 wherein the at least one visual stimulus is delivered with background light.

12. The method of claim 1 wherein the stimulus is delivered to less than the full field of vision of the test subject.

13. A method for performing a full-field stimulus threshold (FST) test on a test subject, the method comprising:
delivering a visual stimulus to at least one eye of the test subject, wherein the visual stimulus comprises a plurality of flashes of light, wherein each of the plurality of flashes of light has an intensity and a duration;
obtaining at least one response from the test subject, wherein the at least one response corresponds to whether the test subject perceived at least one of the plurality of flashes of light; and
determining the lowest intensity of light that the test subject is able to perceive based on the at least one response from the test subject.

14. The method of claim 13 wherein the frequency of the plurality of flashes of light ranges from 1 hertz to 120 hertz.

15. The method of claim 13 wherein the duration of each individual flash of light ranges from 0.25 milliseconds to 30 milliseconds.

16. The method of claim 13 wherein the plurality of flashes of light are delivered in at least two different colors of light.

17. The method of claim 13 wherein the plurality of flashes of light are delivered with no background light.

18. The method of claim 13 wherein the plurality of flashes of light are delivered with background light.

19. The method of claim 13 wherein each of the plurality of flashes of light are separated by a fixed period of time.

20. The method of claim 13 wherein four millisecond flashes of light are delivered at a rate of 10 hertz for a period of 200 milliseconds.

21. The method of claim 13 wherein three 4 millisecond flashes of light are delivered to the at least one eye of the test subject.

22. The method of claim 13 wherein the intensity of the plurality of flashes of light is adjusted over the duration of the flash of light.

23. The method of claim 13 wherein the plurality of flashes of light are delivered to both eyes of the test subject.

24. The method of claim 23 wherein the plurality of flashes of light are delivered simultaneously to both eyes of the test subject, with one eye receiving a stimulus of a first intensity and first duration and the second eye receiving a stimulus of a second intensity and second duration.

25. The method of claim 13 wherein the stimulus is delivered to less than the full field of vision of the test subject.

26. A method for performing a full-field stimulus threshold (FST) test on a test subject, the method comprising:
delivering at least one visual stimulus to at least one eye of the test subject, wherein the at least one visual stimulus comprises a flash of light having an intensity and a duration; wherein the at least one visual stimulus is delivered with background light, and further wherein a single controller is used to control the delivery of the at least one visual stimulus and the delivery of the background light;
obtaining at least one response from the test subject, wherein the at least one response corresponds to whether the test subject perceived the at least one visual stimulus; and
determining the lowest intensity of light that the test subject is able to perceive based on the at least one response from the test subject.

27. The method of claim 26 wherein the single controller is configured to adjust at least one of intensity and duration of the at least one visual stimulus and the background light.

28. The method of claim 26 wherein the at least one visual stimulus comprises a plurality of flashes of light.

29. The method of claim 28 wherein the plurality of flashes of light comprises different colors light.

30. The method of claim 26 wherein the intensity of the flash of light is adjusted over the duration of the flash of light.

31. The method of claim 26 wherein the background light is presented at a luminance level selected from the group consisting of 30 cd/m², 100 cd/m² 1,000 cd/m² and 10,000 cd/m².

32. The method of claim 26 wherein the color of the background light is different from the color of the flash of light.

33. The method of claim 26 wherein the color of the background light is the same as the color of the flash of light.

34. The method of claim 26 wherein the at least one visual stimulus is delivered to both eyes of the test subject.

35. The method of claim 34 wherein the at least one visual stimulus is delivered simultaneously to both eyes of the test subject, with one eye receiving a stimulus of a first intensity and first duration and the second eye receiving a stimulus of a second intensity and second duration.

36. The method of claim 26 wherein the stimulus is delivered to less than the full field of vision of the test subject.

37. A method for performing a full-field stimulus threshold (FST) test on a test subject, the method comprising:
delivering a first visual stimulus to a first eye of the test subject and a second visual stimulus to a second eye of the test subject, wherein the first visual stimulus and the second visual stimulus are provided by different stimulators;
obtaining at least one response from the test subject, wherein the at least one response corresponds to whether the test subject perceived the first visual stimulus or the second visual stimulus; and
determining the lowest intensity of light that the test subject is able to perceive, for at least one eye, based on the at least one response from the test subject.

38. The method of claim 37 wherein a first stimulator is used to deliver the first visual stimulus to the first eye of the test subject and a second stimulator is used to deliver the second visual stimulus to the second eye of the test subject.

39. The method of claim 37 wherein the first visual stimulus is delivered simultaneously with the second visual stimulus.

40. The method of claim 37 wherein the first visual stimulus is delivered before the second visual stimulus.

41. The method of claim 37 wherein at least one of the first visual stimulus and the second visual stimulus comprises at least one flash of light with an intensity that is adjusted over the duration of the flash of light.

42. The method of claim 37 wherein at least one the first visual stimulus and the second visual stimulus comprises a plurality of flashes of light.

43. The method of claim 37 wherein at least one of the first visual stimulus and the second visual stimulus are delivered with background light.

44. The method of claim 37 wherein the stimulus is delivered to less than the full field of vision of the test subject.

45. A method for performing a full-field stimulus threshold (FST) test on a test subject, the method comprising:
delivering at least one visual stimulus to at least one eye of the test subject, wherein the at least one visual stimulus is delivered to less than the full field of vision;
obtaining at least one response from the test subject, wherein the at least one response corresponds to whether the test subject perceived the at least one visual stimulus; and
determining the lowest intensity of light that the test subject is able to perceive based on the at least one response from the test subject.

46. The method of claim 45 wherein the field of vision is less than the full field of vision but greater than two degrees of the full field of vision.

47. The method of claim 45 wherein the intensity of the plurality of flashes of light is adjusted over the duration of the flash of light.

48. The method of claim 45 wherein a plurality of flashes of light are delivered to the test subject.

49. The method of claim 45 wherein the plurality of flashes of light are delivered with background light.

50. The method of claim 45 wherein the at least one visual stimulus is delivered simultaneously to both eyes of the test subject, with one eye receiving a stimulus of a first intensity and first duration and the second eye receiving a stimulus of a second intensity and second duration.

51. A method for performing a full-field stimulus threshold (FST) test on a test subject, the method comprising:
delivering a visual stimulus to at least one eye of the test subject, wherein the visual stimulus comprises a plurality of flashes of light, wherein each of the plurality of flashes of light has an intensity and a duration, wherein the intensity varies between each of the flashes of light, and further wherein the difference in the intensity between two flashes of light is different from the difference in the intensity between two other flashes of light;
obtaining at least one response from the test subject, wherein the at least one response corresponds to whether the test subject perceived the at least one visual stimulus; and
determining the lowest intensity of light that the test subject is able to perceive based on the at least one response from the test subject.

52. The method of claim 51 wherein the difference between the intensity comprises increments varying between 0.15 Log cd*s/m² and 0.5 Log cd*s/m².

53. The method of claim 51 wherein the plurality of flashes of light are delivered with background light.

54. The method of claim 51 wherein the at least one visual stimulus is delivered simultaneously to both eyes of the test subject, with one eye receiving a stimulus of a first intensity and first duration and the second eye receiving a stimulus of a second intensity and second duration.

55. A method for performing a full-field stimulus threshold (FST) test on a test subject, the method comprising:
delivering a visual stimulus to at least one eye of the test subject, wherein the visual stimulus comprises a plurality of flashes of light, wherein each of the plurality of flashes of light has an intensity and a duration, wherein the duration of time varies between each of the flashes of light, and further wherein the difference in the duration of time between two flashes of light is different from the difference in the duration of time between two other flashes of light;
obtaining at least one response from the test subject, wherein the at least one response corresponds to whether the test subject perceived the at least one visual stimulus; and
determining the lowest intensity of light that the test subject is able to perceive based on the at least one response from the test subject.

56. The method of claim 55 wherein the difference in the duration between the flashes of light is 2.5 seconds and the difference in the duration between the other flashes of light is five seconds.

57. The method of claim 55 wherein the plurality of flashes of light are delivered with background light.

58. The method of claim 55 wherein the at least one visual stimulus is delivered simultaneously to both eyes of the test subject, with one eye receiving a stimulus of a first intensity and first duration and the second eye receiving a stimulus of a second intensity and second duration.

59. Apparatus for performing a full-field stimulus threshold (FST) test on a test subject, the apparatus comprising:
a stimulator for delivering at least one visual stimulus to at least one eye of the test subject, wherein the at least one visual stimulus comprises at least one flash of light, wherein the at least one flash of light has an intensity, a duration and a color;
a controller for controlling the intensity, the duration and the color of the at least one flash of light;
a test subject input device for obtaining at least one response from the test subject, wherein the at least one response corresponds to whether the test subject perceived the at least one visual stimulus; and
a processor for determining the lowest intensity of light that the test subject is able to perceive based on the at least one response from the test subject.

60. The apparatus of claim 59 wherein the controller is configured to perform at least one from the group consisting of:
adjusting the intensity of the flash of light over the duration of the flash of light;
delivering a plurality of flashes of light;
delivering at least one flash of light with background light;
delivering a first visual stimulus to a first eye of the test subject and a second visual stimulus to a second eye of the test subject, wherein the first visual stimulus and the second visual stimulus are different;
delivering the least one visual stimulus to less than the full field of vision of the test subject;
delivering a plurality of flashes of light, wherein the intensity varies between each of the flashes of light, and further wherein the difference in the intensity between two flashes of light is different from the difference in the intensity between two other flashes of light; and
delivering a plurality of flashes of light, wherein the duration varies between each of the flashes of light, and further wherein the difference in the duration between two flashes of light is different from the difference in the duration between two other flashes of light.

61. The apparatus of claim 59 wherein the stimulator comprises at least one light source.

62. The apparatus of claim 61 wherein the light source is one from the group consisting of a ganzfeld bowl stimulator and a flash paddle.

63. The apparatus of claim 59 wherein the lowest intensity of light is calculated using a Weibull function.

64. The apparatus of claim 59 wherein the visual stimulus is delivered to one eye of the test subject.

65. The apparatus of claim 59 wherein the visual stimulus is simultaneously delivered to both eyes of the test subject.

66. The apparatus of claim 65 wherein a first stimulus is delivered to one of eye of the test subject and a second stimulus is delivered to the other eye of the test subject.

67. The apparatus of claim 66 wherein the apparatus comprises a first stimulator for delivering the first stimulus and a second stimulator for delivering the second stimulus.

68. The apparatus of claim 67 wherein the first stimulus is different than the second stimulus.

69. The apparatus of claim 59 wherein the stimulus is delivered to the full field of vision of the test subject.

70. The apparatus of claim 59 wherein the stimulus is delivered to less than the full field of vision of the test subject.
